(19) United States
(12) Patent Application Publication
(10) Pub. No.: US 2006/0018785 A1
(43) Pub. Date: Jan. 26, 2006

(54) Apparatus and methods for variably sterilizing aqueous liquids

(76) Inventor: John G. Bowen, San Dimas, CA (US)

Correspondence Address: Mr. John G. Bowen, P.O. Box 4343, San Dimas, CA, 91773, US

(21) Appl. No.: 11/210,217

(22) Filed: Aug. 22, 2005

Related U.S. Application Data
(63) Continuation of application No. 10/626,373, filed on Jul. 24, 2003.

(51) Int. Cl. A61L 2/04

(52) U.S. Cl. 422/1; 422/307

(57) ABSTRACT

A flow through aqueous liquid sterilization apparatus for controllably providing predetermined values of SAL aqueous liquids being processed through the apparatus.

This U.S. Patent Application is a Continuation of U.S. patent application Ser. No. 10/626,373, titled APPARATUS AND MEHTODS FOR VARIABLY STERILIZING AQUEOUS LIQUIDS, and filed Jul. 24, 2003.

### FIELD OF INVENTION

This invention relates to apparatus and methods by which aqueous (water based) liquids are decontaminated for culinary and other uses, such as for medical purposes. This invention is further related to methods which may decontaminate such aqueous liquids without use of chemical or light energy processes.

### BACKGROUND OF THE INVENTION

There is an ever increasing need for new, more effective, efficient and lower cost methods for decontaminating water and other water based (aqueous) liquids. A profound example of changes in methods of water purification is a new water treatment plant located in Salt Lake City, Utah. Rather than chlorine, this plant employs ozone and ultraviolet light, as ultraviolet light is more effective than chlorine in terms of decontaminating water containing cryptosporidium and other chlorine resistant microbes. However, use of light is known to sometimes be ineffective and at other times be unpredictable when used in water that has variable light transmission quality.

While decontamination and purification are terms generally considered in an ultimate context of complete elimination of any and all undesirable contaminants, it is generally known, as disclosed on page 68 of Principles and Methods of Sterilization, 2^{nd} Edition, published by Charles C. Thomas, Springfield, Ill., in 1983, that complete sterilization should never be considered as completely attained. Rather, biological contaminants should be considered to be eliminated logarithmically, such as being measured by time constants dependent upon intensity and method of treatment. As an example, if a process kills a particular organism at a rate of 90% per minute, 10% of the organism survives at the end of the first minute of treatment. One percent survives the second minute of treatment and to achieve a kill of 99.9999% requires a treatment period of six minutes.

To codify a standard for sterilization, the Association for the Advancement of Medical Instrumentation (AAMI), 110 N. Glebe Road, Suite 220, Arlington, Va. 22201-4795 has issued a proposed standard for selecting appropriate Sterility Assurance Levels (SALs)(See Proposed Standard on Selecting Appropriate Sterility Assurance Levels published as an Internet bulletin on Feb. 10, 2000). While, SALs are generally used to determine levels of sterilization for medical products, a similar standard may be considered for water and other aqueous liquid purification, as well. AAMI reports, as examples, that items which come into contact with skin may need only an SAL of 10⁻³ while implants or sterile liquid pathway products should be sterilized to an SAL of 10⁻⁶.

Similar considerations might be applied to water purification. Drinking water from one source might be sufficiently pure at an SAL of 10⁻³ while another source might require an SAL of 10⁻⁴ or better. It may also be desired to have a single water purification or sterilization system which could be used for various purposes (e.g. for drinking water or for a medical application). Also, such aqueous liquids as milk might require different sterilization for different packaging and storage requirements. This invention is meant to fulfill a variety of applications related to meeting requirements for a variety of sterilization levels.

### BRIEF SUMMARY AND OBJECTS OF THE INVENTION

In brief summary, this novel invention alleviates all of the known problems related to safely and efficaciously decontaminating aqueous liquids for a variety of uses. In one embodiment, the invention is a “flow-through” device which receives influent contaminated liquid or impure liquid of questionable pollution and provides a sterilized effluent product which may be variably decontaminated to meet a variety of applications. Further, sterilization levels (i.e. SALs) may be facilely, accurately, predictively and variably controlled, depending upon known or assumed characteristics of an influent liquid to be sterilized and projected use of that liquid.

The invention comprises a liquid source, a flow and pressure controller which provides a variable control setting for both flow and pressure of influent liquid. From the source, liquid is distributed via closed reservoir (e.g. coils) within a heating chamber. The reservoir has a capacity which holds a liquid volume at least equal to a given maximum desired flow rate for a time necessary for sterilizing the liquid to a predetermined SAL value.

Strategically disposed in thermal communication with the reservoir is a temperature sensor which is used to assure that liquid flowing through the heating chamber is at least at a temperature which is consistent with a desired sterilization temperature. Of course, the controlled flow rate determines dwell time in the heating chamber and, therefore, an ultimate SAL value of effluent liquid streaming from the heating chamber.

Heating should be accurately controlled and may be performed by such heat sources as electric elements, gas burners, solar and/or geothermal energy. To assure that heating is sufficiently accurately controlled, it is preferred that the heating chamber provide an accurately controlled temperature “bath” through which the liquid flows. Presently, an oven filled with a paraffin having a predetermined melting temperature is employed to maintain a precise oven temperature.

Actual sterilization efficiency is dependent upon maintaining a liquid temperature above 100° Centigrade (e.g. 150° Centigrade) at a pressure (e.g. 50 psi) which assures achieving a desired SAL as liquid flows through the reservoir. Depending upon a preprogrammed dwell time in the reservoir, sterilization temperature may be variably determined to achieve a desired SAL. Alternately, and preferably, flow rate may be varied to achieve a target SAL.

Other than flow control at the source or influent site of the reservoir, two other flow control elements are employed. Downstream, near the effluent site of the reservoir, a pressure relief valve is disposed in the effluent flow path to guarantee that a predetermined minimum upstream pressure is maintained within the reservoir. Another, second, valve is also serially disposed in the flow path, preferably distal from the oven and the pressure release valve.

The second valve is selectively gated by an “AND” combination of water temperature and pressure sensors. The temperature and pressure sensors are each disposed at individual predetermined strategic sites within the water flow path. In one embodiment, temperature is sensed by a bi-metallic sensor switch disposed within the reservoir, sufficiently close to the influent site of the reservoir to assure that a predetermined minimum sterilization temperature has been achieved, thereby assuring maintenance of the minimum sterilization temperature within the remainder of the reservoir. In this embodiment, a pressure sensor, having a pressure-sensitive switch, is disposed downstream from the reservoir. The pressure sensor is selectively closed when a predetermined sterilization upstream pressure is detected. The contacts of the temperature sensor and pressure sensor are connected in series such that when contacts of each switch close the second valve is opened (i.e. before the second valve opens, the temperature sensor must sense at least a predetermined temperature and the pressure sensor, likewise, must have detected a predetermined pressure.)

Also, each switch of each sensor are opened and closed at different values (of temperature and pressure), thereby creating a hysteresis in each switching parameter and, as a result, assuring stable operation. For example, the temperature sensor may operate to close the temperature switch at a temperature of substantially 150° and operate to open the switch at 140°. In tandem with the temperature sensor, the pressure sensor may operate to close the pressure switch at 80 psi and open the pressure switch at 50 psi. Only when both switches are closed is the second valve opened.

To preserve as much energy as possible, it is preferred to steer oven effluent through a heat exchanger which transfers heat from the effluent to the oven influent such that temperature, and therefore thermal energy, of liquid flowing from the second valve is substantially reduced. In this manner, by controlling dwell time in the reservoir (in the oven) by controlling liquid flow within predetermined limits, liquid of a desired SAL is provided as a cooled continuous flow effluent product.

Accordingly, it is a primary object to provide an efficacious aqueous liquid purification system which controllably sterilizes aqueous liquid to a predetermined SAL.

It is a fundamental object to provide an aqueous liquid purification system which controllably sterilizes an aqueous liquid to a predetermined level by controlling one or more of:
(a) rate of flow of the aqueous liquid through said system,
(b) temperature at which the aqueous liquid is sterilized and/or
(c) pressure at which the liquid is maintained throughout sterilization.

It is an important object to provide a system which inherently maintains a predetermined pressure in a heating unit thereby assuring that aqueous liquid in the heating unit is maintained in a substantially liquid state while being sterilized therein.

It is an object to provide a process by which a heating unit, through which aqueous liquid flows and in which the aqueous liquid is heated, is maintained at a precise temperature.

It is an object to provide a system which assures a predetermined pressure of effluent flowing from said system.

It is an object to provide an energy efficient system which transfers energy from effluent liquid, after sterilization, to influent liquid before sterilization, thereby reducing effluent temperature to a predetermined safer lower temperature level before leaving the system and preheats influent aqueous liquid before it enters the heating unit.

These and other objects and features of the present invention will be apparent from the detailed description taken with reference to accompanying drawings.

### BRIEF DESCRIPTION OF THE DRAWINGS

FIG. 1 is schematic of an aqueous liquid sterilization system which may be adjusted to control degree of purification (SAL).

FIG. 2 is a schematic of a test system model used to determine effectiveness of a sterilization process consistent with the instant invention.

### DETAILED DESCRIPTION OF THE ILLUSTRATED EMBODIMENTS

In this description, the term proximal is used to indicate nearness of a referenced item to the object of the sentence describing its position. The term distal should be interpreted as indicating “away from” a referenced item. Numbers and primes of the same numbers are used to indicate items of related mechanics and function, but which may have physical differences.

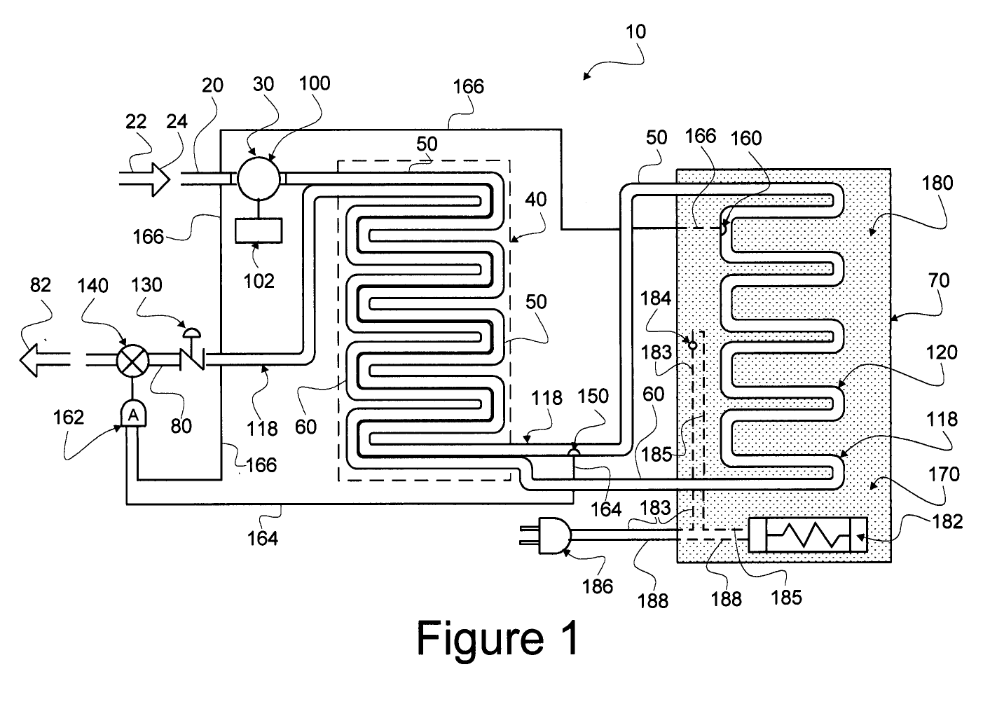

Reference is now made to the embodiment illustrated in FIG. 1. While only a single embodiment is provided herein, it should be apparent to one skilled in water and other aqueous liquid purification by sterilization that other embodiments may be employed within the scope of the invention.

As seen in FIG. 1, a water sterilization system 10 comprises an influent channel 20, wherethrough water from a source 22 (see arrow 24) is delivered, a pump subsystem 30, a heat exchanger 40 through which influent liquid flows in an input pathway 50 and through which effluent liquid flows in an output pathway 60, a pressurized heating chamber 70 and a discharge pathway 80 (see arrow 82).

Pump subsystem 30 comprises a pump 100 and a pump controller 102. Pump 100 should have a variable pumping capacity to supply a predetermined volume of liquid flow through the system against a back pressure which is the consequence rising of a temperature rise in heating chamber 70 and back-pressure of release valve 130. It should be noted that no pump may be required if pressure of the source exceeds the back pressure. However, in both cases, it is necessary to control flow to assure liquid is retained in heating chamber 70 for a period sufficiently long to achieve a desired SAL. In cases where flow is not pump controlled and upstream pressure is known, a flow restricting orifice may be employed. In those instances where controlled flow rates are used to variably determine SALs of effluent, an adjustable orifice may be employed.

Heat exchanger technology is well known in water heating and cooling art. However, it is important that as much energy as possible be transferred from liquid in output or effluent pathway 60 to input or influent pathway 50 within heat exchanger 40 to minimize heat energy loss. For these reasons, pathway 50 should be proximal to and in good thermal communication with pathway 60.

It is critical that the system liquid pathway 118 (a combination of input pathway 50, an internal heating chamber pathway 120 and output pathway 60) be capable of withstanding an internal pressure generated by heating of liquid within the pathway to a desired temperature while maintaining the liquid state. As an example, liquid at 150° Centigrade has a vapor pressure of 55 pounds per square inch (psi). To assure liquid at 150° does not change state, internal pressure in pathway 118 must exceed 55 psi.

For this reason, a flow resisting element, such as a pop valve 130 is serially connected in a section of output pathway 60 distal from heating chamber 70 and heat exchanger 40. To further assure that there is no flow through pathway 118 (and discharge pathway 80) until conditions for water sterilization have been reached in heating chamber 70, a second valve, numbered 140, is serially connected in discharge pathway 80. In this embodiment, valve 140 is a solenoid valve activated by an AND combination of two switches, a pressure sensor switch 150 and a temperature sensor switch 160.

Each sensor switch (150 and 160) activates to open at a first predetermined level and closes at a second predetermined level. For example, pressure sensor switch 150 may be selected to close at 80 psi and open at 55 psi, while temperature sensor switch 160 may close at 150° centigrade and open at 140° centigrade. As such, switch 150 must sense 80 psi and switch 160 must sense 150° centigrade (symbolized by AND gate 162) to open valve 80 to permit effluent to flow through system 10. Note, pressure sensor switch 150 is connected to AND gate 162 via line 164 and temperature sensor switch 160 is connected to AND gate 162 via line 166.

To sterilize water at least to a predictable SAL, both system 10 water flow rate and heating chamber 70 temperature must be known and well controlled to assure liquid in pathway 120 is resident in heating chamber 70 for a long enough period to assure the desired sterilization level. Water flow rate may be closely controlled by pump 100 and pump controller 102. Temperature is preferably induced in liquid in pathway 120 by a high heat capacity bath 170 which has high heat transfer and precise temperature control characteristics.

While other media may be used in such a bath, such as oil or high heat capacity fluids, it is preferred to use a precisely specified paraffin, such as matter 180. In this case, matter 180 is a stable substance which changes state from a solid to a liquid and maintains a constant desired predetermined temperature during the state change. Particularly suited for use in bath 170 is paraffin. Paraffin may be formulated to accurately and precisely melt at a selected temperature between 100° centigrade and 170° centigrade. Such paraffin is currently available from ASTOR Specialty Chemicals, 160° Commerce, Marshall, Tex. 75670. As an example, matter 180 may be selected to have a melting point of 150° centigrade.

Heating of matter 180 is accomplished by a set of electrical heating elements, generally referenced by 182, which are turned off and on by a bimetallic temperature switch 184. Heating elements 182 are powered by a standard electrical plug assembly 186 which is interconnected to heating elements 182 via electrical lines 183, 185 and 188. Bimetallic temperature switch 184 is interposed between line 183 and line 185. Dashed lines indicate electrical line residence in bath 170.

Switch 184 is selected to open at a temperature which is fractionally above the melting point of matter 180 (e.g. 152° centigrade and to close at a temperature at or just below the melting point of matter 180 (i.e. 150° centigrade). So constrained, heating of matter 180 is the result of a hysteresis effect, making operation stable.

System 10 may be constructed from a large number of parts generally available in commerce today. Examples of parts which may be used are as follows:

System 10 Part Commercial Part
Pump 100 Flojet Pump model #03655E7011A, available from Flojet, ITT Industries, 201 CON, Fort Hill Ranch, Calif.
Temp. Sensor Switch 184 Texas Instruments 20260 bimetal thermal switch, Normally Closed.
Temp. Sensor Switch 160 Texas Instruments 20260 bimeatl thermal switch, Normally Open.
Pres. Sensor Switch 150 Texas Instruments 36PS-50 psi, Normally Open.
Heating Elements 182 TEMCO Finned Strip Heaters, Type 4, 500 Watt, available from TEMCO, 607 North Central, Wood Dale, Ill. 60191.

Valve 140 Solenoid Valve #4639K8 (120 volt, 0.13 Amps), available from McMaster-Carr Supply Co., www.mcmaster.com.
Press. Rel. Valve 130 CA Series In-line Adjustable Relief Valve having a cracking pressure range from 50 to 150 PSIG, available from NUPRO Company, 4800 East 345^{th} Street, Willoughby, Ohio 44094.

Pathway 118 Preferably constructed from high pressure, stainless steel tubing (with all joints welded to withstand temperatures above melting temperature of matter 180).

The time to sterilize an item, using saturated steam at a given temperature is well known and summarized in Table 1 below:

TABLE 1

| | Sterilization |
|---|---|
| Time to sterilize | temperature |
| 20 minutes | 121° Centigrade |
| 10 minutes | 128° Centigrade |
| 3.5 minutes | 134° Centigrade |
| Nearly instantaneous | 141° Centigrade |

However, data in Table 1 is not directly related to SALs. Therefore, some nominal experimentation may be necessary to develop known sterilization criteria for each system 10. Through experimentation it has been found that water sterilization by system 10 at different parametric levels of flow and temperature yields different SALs for assorted species tested. It should not be surprising that SALs vary for different microbes and other water-borne organisms.

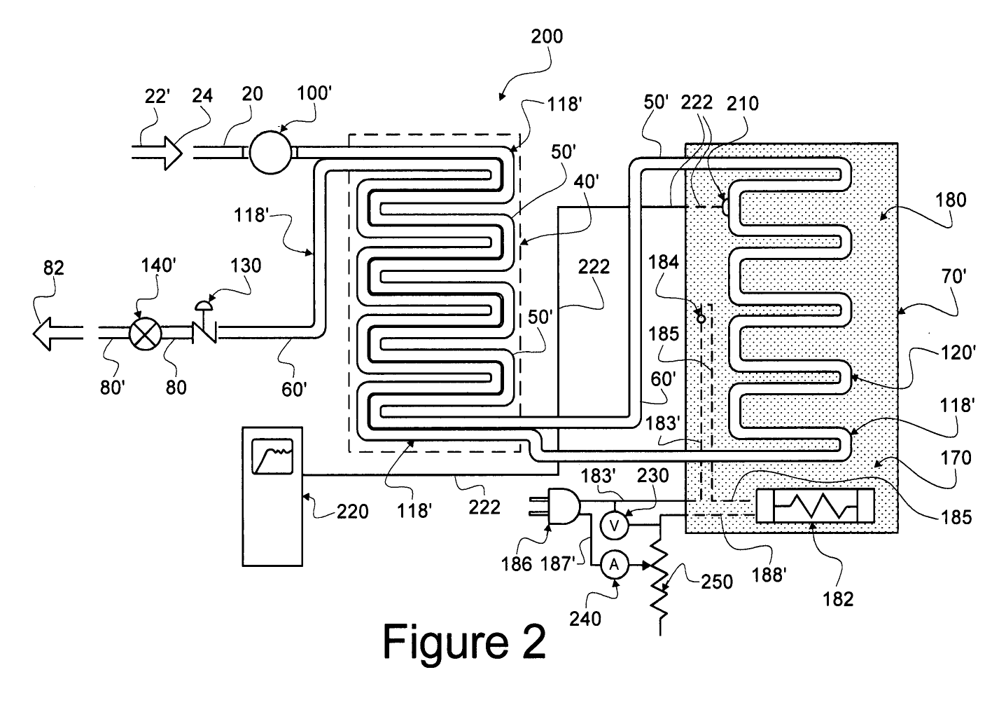

FIG. 2 is a schematic representation of a test model 200 used to test effectiveness of sterilizing aqueous solutions by processes consistent with the instant invention. As seen in FIG. 2, model 200 comprises a source 22′ of influent contaminated water. In this case, source 22′ is a 60 gallon drum strategically disposed above a pump 100′ for easy priming.

Similar to system 10 seen in FIG. 1, model 200 comprises an influent channel 20, wherethrough water from a source 22′ (see arrow 24) is delivered, a pump 100′, a heat exchanger 40′ through which influent liquid flows in an input pathway 50′ and through which effluent liquid flows in an output pathway 60′, a pressurized heating chamber 70′ and a discharge pathway 80′ (see arrow 82).

Pump 100′ is manually controllable. Pump 100′ has a variable pumping capacity which is manually adjusted to supply a predetermined volume of liquid flow through the system. A needle valve 140′ is used for manual control of flow through model 200. Temperature of solution in pathway 120′ (which is the in heating bath portion of total system pathway 118′)is monitored by means of a temperature sensor 210 (a thermocouple) and a graphic recorder 220. Note that an electrical line 222 interconnects sensor 210 and recorder 220. In this model, an Esterline Angus Video Graphic Model B recorder was used.

Energy supplied to heating elements 182 of oven 70′ of model 200 was monitored by a voltmeter 230 and an ammeter 240. Varying amounts of energy was supplied from electrical plug assembly 186 to heating elements 182 and therefrom to bath 170 of oven 70′ via a variable voltage rheostat 250. Note that electrical lines 183′, 185, 187′ and 188′ are used to supply electrical energy to heating elements 182. Line 183′ interconnects assembly 186 and one side of temperature sensor switch 184. The other side of temperature sensor switch 184 is connected to heaters 182 via electrical line 185. Ammeter 240 is placed in series (via electrical line 187′) from plug assembly 186 to rheostat 250. Rheostat 250 is connected to heating elements 182 via electrical line 188′.

Model 200 system liquid pathway 118′ was designed to be capable of withstanding any internal pressure generated by heating of liquid within the pathway to temperatures within the scope of reasonable experimental safety limits while constraining liquids in pathway 118′ to remain in a liquid state.

In model 200, liquid pathway 118′ had a volume of 600 ml. Temperature was held between 143 and 144 degrees centigrade. Pump 100′ supplied liquid at a constant pressure of 95 psi. Heat exchanger 40′ employed coaxial piping. Pop valve 130 (a pressure release valve) was rated at 50 psi. As earlier disclosed, needle valve 140′ was used to manually regulate flow rate through pathway 118′.

Temperature of pathway 118′ was manually monitored by thermocouple 210 placed in thermal communication with pathway 118′. As earlier disclosed, an Estiline Angus model videographic system B (recorder 220) was used to continuously monitor temperature. Variations in temperature caused by increasing or decreasing rate of flow were adjusted by rheostat 250 which adjusted electric power supplied to a set of heating elements, generally referenced as 182. In model 200, four such 500 watt heating elements were employed.

Biologic testing was performed to determine effectiveness of sterilization at different flow rates using water contaminated with the following four different microorganisms:
1. Bacillia sterothermopbilus
2. E. coli
3. Candida Aldicans
4. Pseudomonas aeruginosa

A predetermined quantity of each microorganism was mixed with 25 gallons of distilled water and dispensed into a drum to provide source 22′. A serial dilution of each batch of microorganisms was titrated and tested to establish the concentration of each organism in the batch. Every batch prepared was determined to contain at least 10⁶ organisms.

Each of the four test organisms were run in duplicate on different days. A test protocol was prepared to run five different effective sterilization periods on each organism. Generally, flow rates employed were divided into a plurality of constant flow one and one-half hour periods. In the runs, flow rates used ranged from 50 to 350 milliliters per minute, in 50 milliliter per minute increments. However, due to lack of meaningful results at lower flow rates and limits on volumes of solution available in model 200, less than a complete complement of flow rates were often used, e.g. 250, 300 and 350 milliliters/minute were used in a test run performed on Apr. 25, 2003, results of which are provided hereafter.

Samples were taken at fifteen minute intervals throughout each test period (providing seven samples per period). Each sample was tested by placing a milliliter aliquot onto a blood agar or enriched agar plate, incubated for 48 hours and read by a qualified microbiologist. As seen by the examples of data provided hereafter, kill ratio of each sample generally exceeded a 10⁻⁶ organism reduction in processed effluent.

Though all tests showed similar sterilization results, a summary of two tests using bacillia sterothermopbilus are provided, in Tables 3 and 5 below, as exemplary results of running model 200. Dates of performance of the exemplary tests were Apr. 19, 2003 and Apr. 25, 2003. For each test run, content of source 22′ was titrated as a control. Two sets of such results, one set for each solution tested on Apr. 19, 2003 and Apr. 25, 2003, are provided separately in Tables 2 and 4, respectively.

TABLE 2
Titration of Stock Culture Used 4/19/03
Sample volume is 1.0 ml/each dilution
(unless otherwise noted).
Plates incubated @ 59° C. for 18 hours

| Event | Dilution | Colonies |
|---|---|---|
| 1 | 10⁻¹ | TNTC ** |
| 2 | 10⁻² | TNTC ** |
| 3 | 10⁻³ | TNTC ** |
| 4 | 10⁻⁴ | TNTC ** |
| 5 | 10⁻⁵ | no record |
| 6 | 10⁻⁶ | no record |
| 7 | 10⁻⁷ | 600 |
| 8 | 10⁻⁸ | *** |
| 9 | 10⁻⁹ | *** |
| 10 | 10⁻¹⁰ | *** |
| 11 | 10⁻⁰ * (Stock) | TNTC ** |

* 0.250 ml sample volume
** Too Numerous To Count
*** Titration not performed due to measurable level at event 7

TABLE 3
Test Run 4/19/03 (Temperature of pathway 118′: 143 to 144 ° C.

| | Flow Rate | Time in minutes | |
|---|---|---|---|
| Run # | (ml/min) | (within run) | Colonies |
| I | 100 | 0 | 2 ** |
| | | 15 | 0 |
| | | 30 | 0 |
| | | 45 | 0 |
| | | 60 | 0 |
| | | 75 | 0 |
| | | 90 | 0 |
| II | 150 | 0 | 0 |
| | | 15 | 0 |
| | | 30 | 0 |
| | | 45 | 0 |
| | | 60 | 0 |
| | | 75 | 0 |
| | | 90 | 0 |
| III | 200 | 0 | 0 |
| | | 15 | 0 |
| | | 30 | 0 |
| | | 45 | 0 |
| | | 60 | 0 |
| | | 75 | 0 |
| | | 90 | 0 |
| IV | 300 | 0 | 0 |
| | | 15 | 0 |
| | | 30 * | 450 |
| | | 45 * | 150 |

* See note (reference [*]) following Table 5.
** Initial contamination in effluent pathway.

TABLE 4
Titration of Stock Culture Used 4/25/03
Sample volume is 1.0 ml/each dilution
(unless otherwise noted).
Plates incubated @ 59° C. for 18 hours

| Event | Dilution | Colonies |
|---|---|---|
| 1 | 10⁻¹ | TNTC ** |
| 2 | 10⁻² | TNTC ** |
| 3 | 10⁻³ | 1000 |
| 4 | 10⁻⁴ | 400 |
| 5 | 10⁻⁵ | 250 |
| 6 | 10⁻⁶ | 180 |
| 7 | 10⁻⁷ | 120 |
| 8 | 10⁻⁸ | 75 |
| 9 | 10⁻⁹ | 64 |
| 10 | 10⁻¹⁰ | 25 |
| 11 | 10^{-0 * (Stock)} | TNTC ** |

* 0.250 ml sample volume
** Too Numerous To Count

TABLE 5
Test Run 4/25/03 (Temp. of pathway 118′: 143 to 144 ° C.

| | Flow Rate | Time in minutes | |
|---|---|---|---|
| Run # | (ml/min) | (within run) | Colonies |
| I | 250 | 0 | 0 |
| | | 15 | 0 |
| | | 30 | 0 |
| | | 45 | 0 |
| | | 60 | 0 |
| | | 75 | 0 |
| | | 90 | 0 |
| II | 300 | 0 | 0 |
| | | 15 | 0 |
| | | 30 | 0 |
| | | 45 | 0 |
| | | 60 | 0 |
| | | 75 | 0 |
| | | 90 | 0 |
| III | 350 | 0 | 0 |
| | | 15 | 0 |
| | | 30 | 0 |
| | | 45 | 0 |
| | | 60 | 0 |
| | | 75 * | 1000 |
| * Tank take-off connection at a point 2.5 gallons from tank bottom. Test samples were taken until flow became erratic due to tank drainage. This variation in liquid flow caused oven temperature to first increase rapidly, turning off bimetal over temperature protectors (not otherwise disclosed) resulting in a dramatic decrease in operating temperature. Data at run #III, time 75 minutes (and at run#II, times 30 and 45 minutes) provided to permit a comparative assessment with data | | | |
| # derived from system 200 under normal operating conditions. | | | |

Results from all tests proved the efficacy of the instant invention. Independent of microorganisms tested and flow rates tested, system model 200 clearly sterilized contaminated influent to produce a continuously flowing sterilized effluent. The effectiveness of sterilization was demonstrated when compared with final samples of contaminated and unsterilized effluent which resulted when temperature of model 200 precipitously declined as an end-of-run phenomenon when water from source 22′ was depleted.

The invention may be embodied in other specific forms without departing from the spirit or essential characteristics thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. Flow through aqueous liquid purification apparatus for sterilizing liquid to a target SAL comprising:
a flow through reservoir comprising an input pathway, an output pathway and an internal heating chamber pathway, disposed between the input pathway and output pathway, said internal heating chamber pathway comprising an enclosed, elongated flow path along which liquid is displaced and disposed for being heated to a sterilizing temperature;
each of said input, output and heating chamber pathways comprising sidewalls capable of withstanding increased internal pressure generated by liquid being heated to the sterilizing temperature while being kept in a liquid state;
a heating chamber comprising media which surrounds and is in thermal contact with the internal heating chamber pathway along the enclosed elongated path, said media being heated to a critical predetermined temperature consistent with heating liquid within the heating chamber pathway to the sterilizing temperature;
a source of pressurized liquid, such source providing the liquid to be sterilized within said heating chamber pathway;
a flow resisting element, disposed in the output pathway, which restricts effluent flow such that, in combination with said pressurized source of liquid, the combination maintains a predetermined minimum upstream pressure within the internal heating chamber pathway;
a valve subsystem comprising a temperature sensor switch which is disposed to be activated to an ON state when liquid in the heating chamber pathway is at a first predetermined temperature which is at least as high as the critical predetermined temperature;
said valve subsystem further comprising a pressure sensor switch disposed in the heating chamber pathway which is activated to an ON state when pressure in the heating chamber is at least a first determined pressure consistent with sterilization dynamics; and
said valve subsystem further comprising a valve which is in an OPEN state only when said temperature sensor switch is in an ON state and said pressure sensor switch is in an ON state.

2. The flow through aqueous liquid purification apparatus according to claim 1 further comprising a heat exchanger for exchanging heat between cooler liquid flowing into the input pathway and warmer liquid flowing through the output pathway.

3. The flow through aqueous liquid purification apparatus according to claim 1 wherein the flow resisting element is a pressure relief valve.

4. The flow through aqueous liquid purification apparatus according to claim 1 wherein said source comprises a controller for varying source pressure to thereby provide the liquid flow through the heating chamber pathway at a rate which sterilizes the liquid to the desired SAL.

5. The flow through aqueous liquid purification apparatus according to claim 1 wherein at least one of said temperature sensor switch and said pressure sensor switch is switched to OFF at a sensed level which is less than an associated ON level whereby a hysteresis effect is established for the OPEN state of said valve.

6. The flow through aqueous liquid purification apparatus according to claim 1 wherein said valve subsystem further comprises a combination comprising the valve, the temperature sensor switch, the pressure sensor switch and an AND gate which, in combination, operate to open the valve only when liquid upstream from the valve is decontaminated.

7. The flow through aqueous liquid purification apparatus according to claim 1 wherein said media comprises material which changes from a solid to liquid state at the predetermined temperature.

8. The flow through aqueous liquid purification apparatus according to claim 7 wherein said media comprises paraffin.

9. The flow through aqueous liquid purification apparatus according to claim 1 wherein said liquid pressure and flow source comprises a single pump.

10. The flow through aqueous liquid purification apparatus according to claim 1 wherein said liquid pressure and flow source comprises a static reservoir with a flow control orifice.

11. A method for sterilizing liquid to a target SAL comprising the steps of:
(a) providing a flow through aqueous liquid purification apparatus comprising:
(i) a flow through reservoir comprising an input pathway, an output pathway and an internal heating chamber pathway, disposed between the input pathway and output pathway, said internal heating chamber pathway comprising an enclosed, elongated flow path along which liquid is displaced and disposed for being heated to a sterilizing temperature;
(ii) each of said input, output and heating chamber pathways comprising sidewalls capable of withstanding increased internal pressure generated by liquid being heated to the sterilizing temperature while being kept in a liquid state;
(iii) a heating chamber comprising media which surrounds and is in thermal contact with the internal heating chamber pathway along the enclosed elongated path, said media being heated to a critical predetermined temperature consistent with heating liquid within the heating chamber pathway to the sterilizing temperature;
(iv) a source of pressurized liquid, said source providing liquid to be sterilized within said heating chamber pathway;
(v) a flow resisting element, disposed in the output pathway, which restricts effluent flow such that, in combination with said pressurized source of liquid, maintains a predetermined minimum upstream pressure within the internal heating chamber pathway;
(vi) a valve subsystem comprising a temperature sensor switch which is disposed to be activated to an ON state when liquid in the heating chamber pathway is at a first predetermined temperature which is at least as high as the critical predetermined temperature;
(vii) said valve subsystem further comprising a pressure sensor switch disposed in the heating chamber pathway which is activated to an ON state when pressure in the heating chamber pathway is at least a first determined pressure consistent with sterilization dynamics; and
(viii) said valve subsystem further comprising a valve which is in an OPEN state only when said temperature sensor switch is in an ON state and when said pressure switch is in an ON state;
(b) delivering liquid into the apparatus at a rate which is consistent with a desired SAL value for effluent from the apparatus, liquid being delivered via said source into the input, heating chamber and output pathways;
(c) applying heat to the heating chamber pathway via the heating chamber;
(d) opening the valve to permit effluent flow of sterilized liquid only when the temperature sensor switch is in an ON state and the pressure sensor switch is in an ON state.
